(19) United States
(12) Patent Application Publication
(10) Pub. No.: US 2018/0153638 A1
(43) Pub. Date: Jun. 7, 2018

(54) BODY SUPPORT DEVICE FOR SURGERY

(71) Applicant: FACULDADE DE ARQUITETURA, Lisboa (PT)

(72) Inventor: Joana Leticia PAIXAO CORREIA, Porto Santo (PT)

(21) Appl. No.: 15/736,085

(22) PCT Filed: Jun. 9, 2016
(86) PCT No.: PCT/IB2016/053400

§ 371 Date: Dec. 13, 2017

(30) Foreign Application Priority Data
Jun. 13, 2015 (PT) 108550

(51) Int. Cl. A61B 90/60; A61G 15/10

(52) U.S. Cl. CPC A61B 90/60; A61B 2017/00973; A61G 15/10

(57) ABSTRACT

The present application discloses a body support device for surgery with a dynamic support for the torso. This device stimulates the adoption of a more natural posture and provides the dissipation of the load placed on the bone and muscle structures in the spinal column through the combination of the torso support (1) to the posterior support (13). The posterior support (13) consists of the systematization of the human vertebral column, comprising a set of segments (13′) chained and aligned by the passage of an elastic medium (14) therein, controlled by the user's action. Thus, this technology allows for minimizing surgeon's physical fatigue resulting from a prolonged posture in surgery.

### TECHNICAL FIELD

The present application discloses a body support device for use in surgery.

### BACKGROUND

Surgery is conducted in circumstances characterized by the demand for maximum concentration and a stable and prolonged position is necessary. Many times, the adopted postures provoke discomfort due to the bad equipment existing solutions. One of the problems observed during surgical procedures is related to the surgeon's body posture. The adoption of certain positions for a long period of time, often overlooked and neglected, is the source of small problems at the muscular structure level, initially characterized by discomfort and later by the pain they cause to the surgeon. There are studies that conclude that these problems are, in the long-term, responsible for the alteration and deterioration of bone and muscle structures.

The solutions available on the market addressing this issue for surgeons lack efficient support for the torso and, at the same time, the freedom to perform the necessary movements for the surgery. It is essential for the surgeon to maintain proximal stability to arrange a good distal mobility and, even so, carry out small postural adjustments throughout the surgery. Some surgeons opt for the use of lumbar back braces or dorsal vests in addition to the existing surgical seats. These solutions, despite minimizing some symptoms, do not provide the necessary dorsal and lumbar support nor the dissipation of the load placed on the bone and muscle structures of the spinal column. In surgery, the muscle is required to maintain a long-lasting performance with great accuracy, but low intensity, requiring some resistance in detriment of force. Thus, it is inevitable that the effects of the gravity are felt on the postural muscles and, later, on the bone structures, such that it is essential to have a surface to witch the experienced load can be transferred.

### SUMMARY

The present application discloses a body support device for surgery with a dynamic support for the torso. This device stimulates the adoption of a more natural posture and provides the dissipation of the load placed on the bone and muscle structures in the spinal column through the combination of the torso support to the posterior support. The posterior support consists of the systematization of the human spine, comprising a set of segments chained and aligned by the passage of an elastic medium therein, controlled by the user's action. Thus, this technical solution allows for minimizing surgeon's physical fatigue resulting from a prolonged posture in surgery.

### BRIEF DESCRIPTION

The present application discloses a body support device with lumbar or lumbar and dorsal support, which allows for performing rotation movements, flexion and lateral extension as well as the adjustment of tension placed on the torso support (1). The height and inclination of the seat (3) are controlled through the activation of two linear actuators, called linear actuator A (17) and linear actuator B (18).

The tension placed on the torso support (1) is controlled by an engine (15), allowing for millimetric adjustments in the user's posture. The device permits attaching the torso support (1) to the posterior support (13) with great accuracy and promotes the adoption of a dynamic posture. The referred torso support (1) was previously placed and attached to its user by a vest (2) by means of a removable attachment. Through this application, it is intended as vest (2) the piece that covers the user's torso.

The shape of the posterior support (13), inspired by the human spine, is characterized by the mobility of each of the constituent elements. Similar to the biological element behind this device's concept, the posterior support (13) is comprised by a chain of segments (13′) that describe a curve adjusted by the operator and whose dimensions are larger in the base, narrowing at the top.

The segments (13′) of the posterior support (13) have a concave spherical cap (female) on its top and a convex spherical cap (male) on the base, which allow the sequential linking thereof. The spherical fittings adopt variable depths according to the dimension of each one of the segments (13′) and provide a variety of movements that include flexion, lateral extension and rotation. The width of the flexion of the torso support (1), when attached to the posterior support (13), as well as its lateral extension varies between 0° and 15°. The shape of the segments (13′) prevents torso extension in the sagittal plane. The range of rotation can vary between 0° and 21°, to the left or to the right.

The segments (13′) are perforated longitudinally, with two holes aligned that house an elastic medium (14) in their interior acting as an aggregating and regulating element of the posterior support (13). The end of the elastic medium (14) is attached to a shaft (5) positioned in the seat support (4). This shaft (5), in turn, is connected to an engine (15) through a belt (23) and, when activated by the tensioner pedal (19) located under the seat (3), it allows for adjusting the tension millimetrically. The elastic medium (14) also allows for realigning the segments (13′) whenever movement is indicated. The torso support (1) is attached to the posterior support (13) through means of a non-permanent connection, rendered in this device through a magnetic mechanism sensible to the passage of electrical power in the bigger dimensions surface of the posterior support (13), activated by pressing attach/release pedal (10). The applied tension on torso support (1) is regulated through the tensioner pedal (19), located in the base (11). The mobility characteristics described, the controlled autonomy of each of the pieces and the tension regulation placed by the elastic medium (14) allow for adjusting the shape of the curvatures according to the operator's needs. When attached, the device allows its user to adopt postures that keep their curvatures as close as possible to the natural curvatures of the spine and permitting the recommended postural adjustments. At the same time, the device provides a load dissipation surface, relieving the user's bone and muscle structures of the effects of the gravity experienced, minimizing the symptoms of fatigue.

The height and inclination of the seat surface (3) are controlled by the user through a lever (16) positioned under the same. This lever (16) activates the two actuators installed in the base (11) of the device, the linear actuator A (17) and the linear actuator B (18). By pressing the lever (16) down, it makes the seat surface go down (3), by pressing up it goes up. The lever (16), when moved forward, tilts the seat surface (3) forward up to an inclination of 5° in relation to the transverse plane. The opposite movement makes the seat surface (3) return to the starting position. The device is controlled independently by the user through the pedals on the base (11). The user controls the action of the engine (15) through the pressure placed by their lower limbs on the tensioner pedal (19) and the attach/release pedal (10), allowing one to maintain complete freedom of the upper limbs. The tensioner pedal (19) allows the tension or distension of the torso support (1) millimetrically, while the attach/release pedal (10) allows the attachment and/or release of the torso support (1) to the posterior support (13). The safety switch (9) is responsible for the activation of all the commands available on the device and is associated with a lock programmer after inactivity, preventing accidental adjustments. The safety switch (9) is activated when pressed and emits a beeping sound for a period of time, during which all settings are allowed. Once the beeping sound stops, the adjustment operations are locked and the device maintains the position set by the user, being imperative to reactivate the safety switch (9) for further adjustments.

### BRIEF DESCRIPTION OF FIGURES

Without intent to limit the disclosure herein, this application presents attached drawings of illustrated embodiments for an easier understanding.

FIG. 1 illustrate a representation of the main components of the body support device, where the reference numbers illustrate the following elements:
1—torso support;
2—vest;
3—seat;
4—seat support;
5—shaft;
6—actuator support;
7—hydraulic suspension;
8—battery;
9—safety switch;
10—attach/release pedal;
11—base;
12—caster;
13—posterior support;
13′—segment;
14—elastic medium;
15—engine;
16—lever;
17—linear actuator A;
18—linear actuator B;
19—tensioner pedal;
20—sleeve;
21—fairing A;
22—fairing B.

FIG. 2 illustrates a frontal and back representation of the body support device, where the reference numbers illustrate the following elements:
1—torso support;
2—vest;
3—seat;
9—safety switch;
10—attach/release pedal;
13—posterior support;
13′—segment;
16—lever;
19—tensioner pedal;
20—sleeve;
21—fairing A;
22—fairing B.

FIG. 3 illustrates a side representation with the minimum and maximum seat height of the body support device.

FIG. 4 illustrates a representation of the minimum and maximum seat inclination of the body support device.

FIG. 5 illustrates a representation of the maximum flexion of the posterior support of the body support device.

FIG. 6 illustrates a representation of the maximum lateral extension of the posterior support of the body support device.

FIG. 7 illustrates a representation of the maximum range of rotation of the posterior support of the body support device.

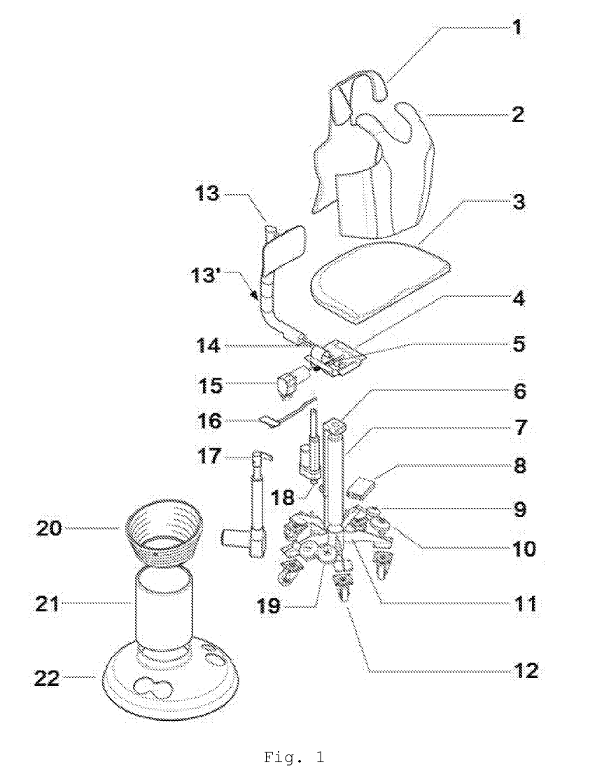

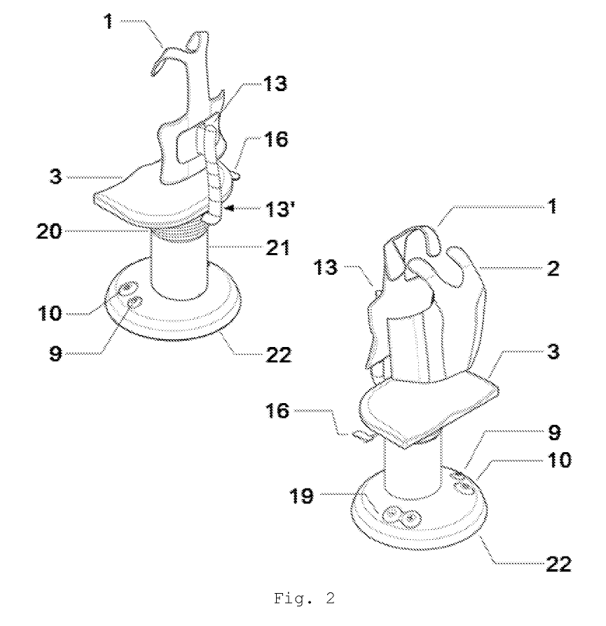

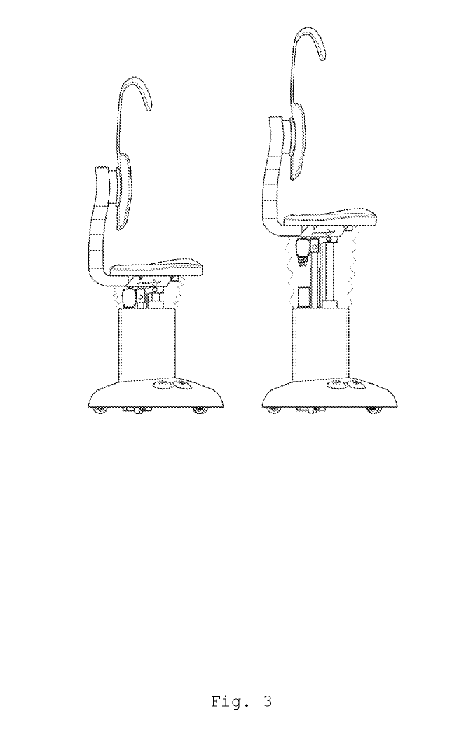

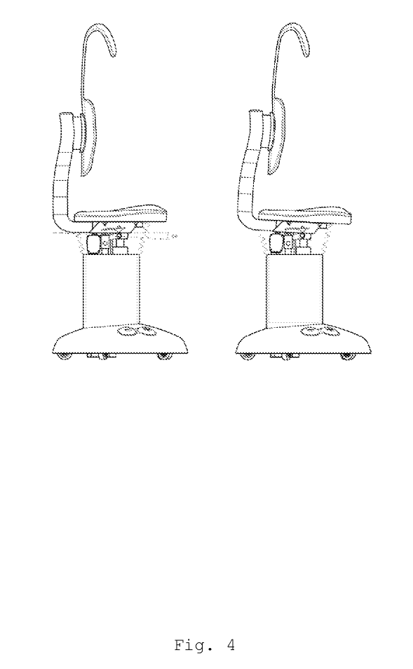

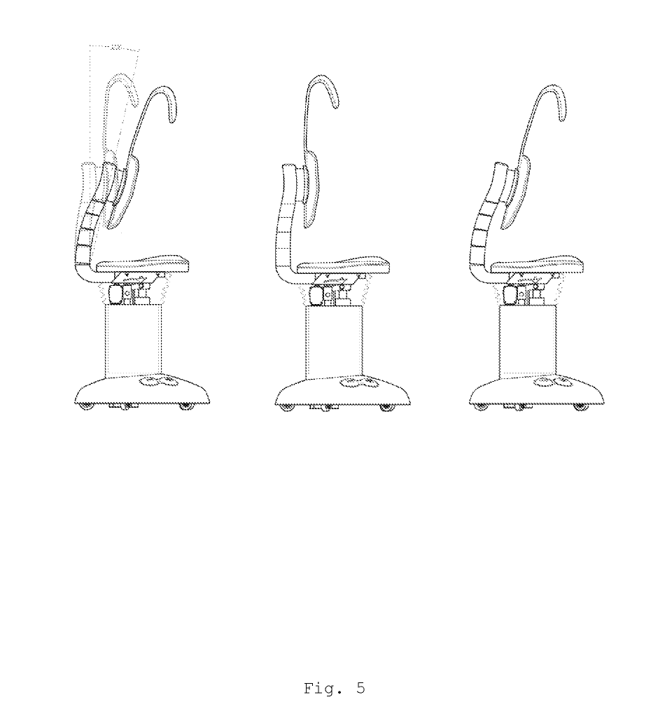

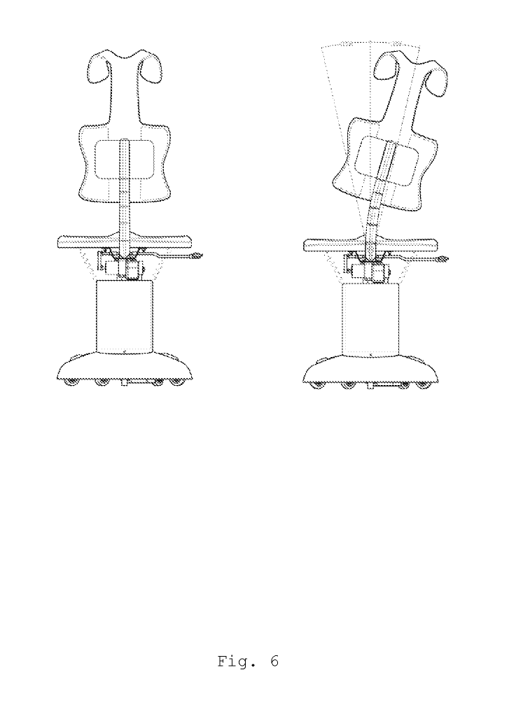

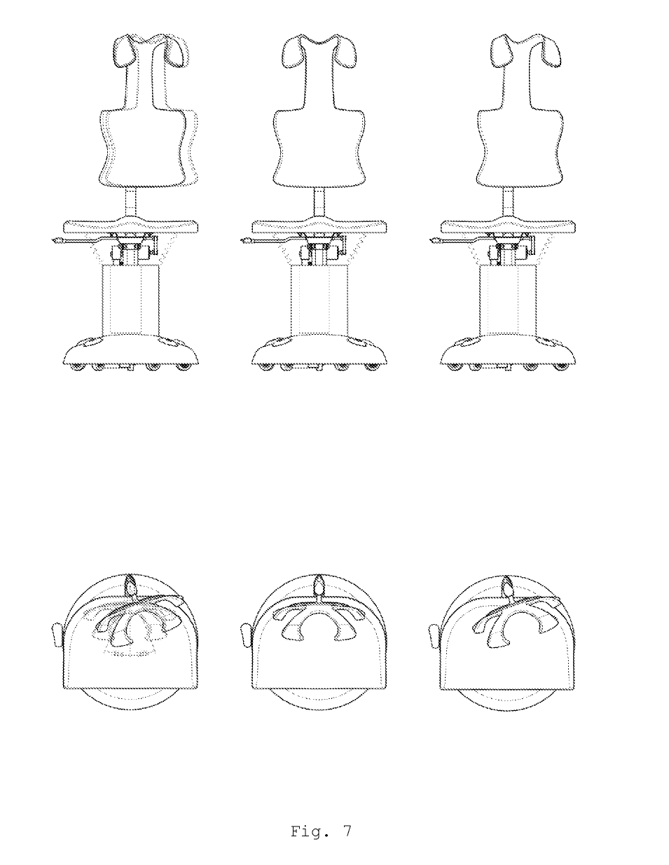

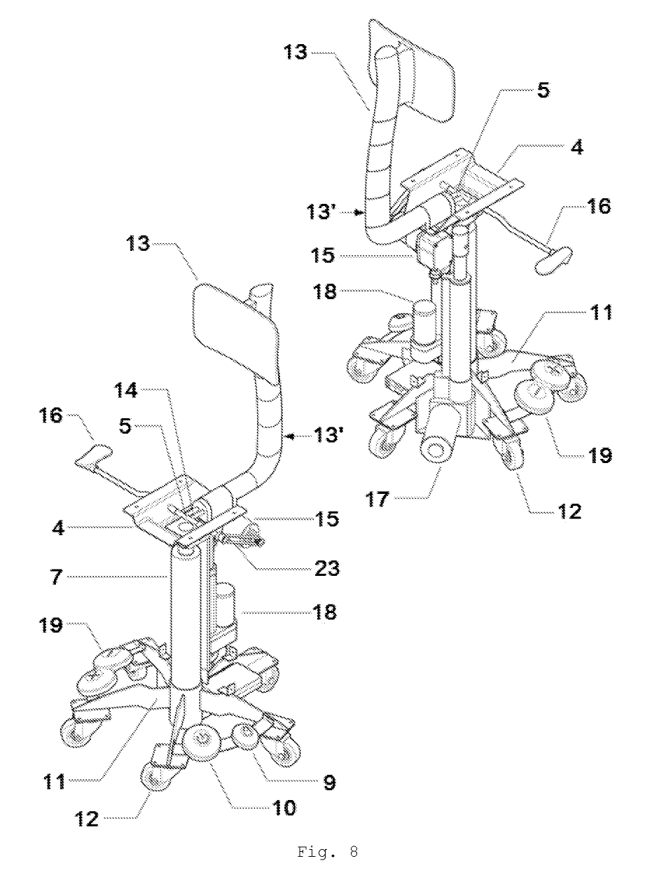

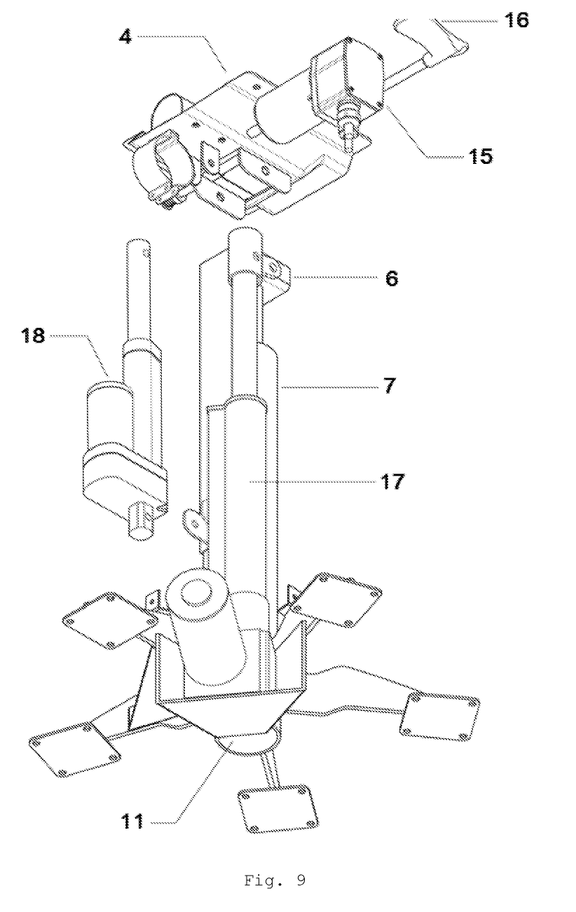

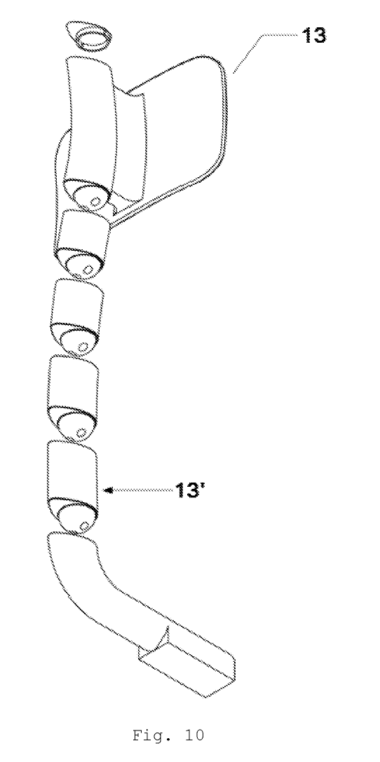

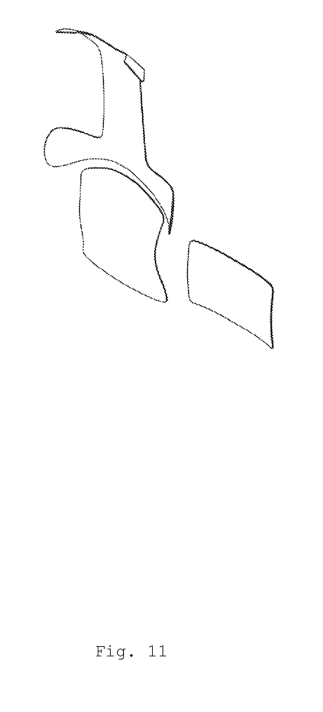

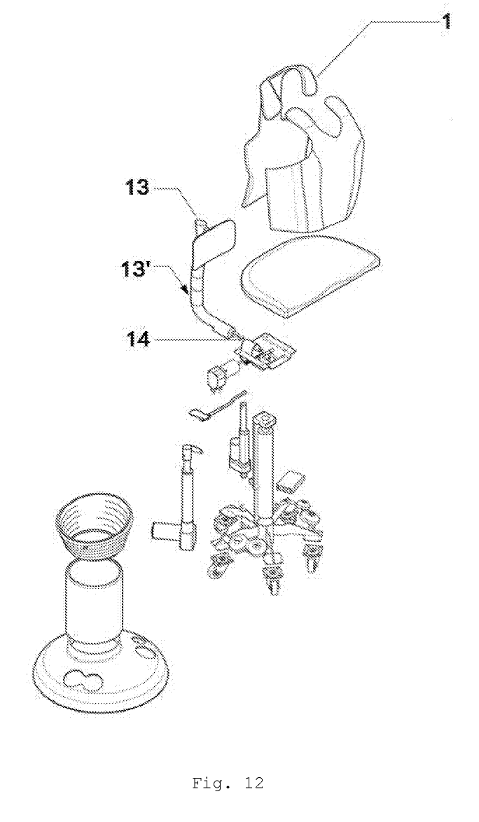

FIG. 8 illustrates a representation of the structure of the body support device with its components, where the reference numbers illustrate the following elements:
4—support;
5—shaft;
7—hydraulic suspension;
9—safety switch;
10—attach/release pedal;
11—base;
12—caster;
13—posterior support;
13′—segment;
14—elastic medium;
15—engine;
16—lever;
17—linear actuator A;
18—linear actuator B;
19—tensioner pedal;
20—sleeve;
21—fairing A;
22—fairing B;
23—belt.

FIG. 9 illustrates a representation in greater detail of the seat, the engine, the lever, actuators, the support for the actuators and the base of the body support device, where the reference numbers illustrate the following elements:
4—seat support;
6—actuator support;
7—hydraulic suspension;
11—base;
15—engine;
16—lever;
17—linear actuator A;
18—linear actuator B.

FIG. 10 illustrates a representation of the posterior support of the body support device, where the reference numbers illustrate the following elements:
13—posterior support;
13′—segment.

FIG. 11 is a representation of the structure the body support of the body support device.

FIG. 12 illustrates the main components of the body support device, where the reference numbers illustrate the following elements:
1—torso support;
13—posterior support;
13′—segment;
14—elastic medium.

### DESCRIPTION OF EMBODIMENTS

Referring to the drawings, herein are described an optional embodiment in more detail, which however is not intended to limit the scope of the present application. The embodiment refers to the use of the body support device in ophthalmological surgeries in operating room.

The present technology discloses one torso support (1) which is placed on the surgeon's torso, allowing freedom of movement of the upper limbs. The shape of torso support (1) is designed from scanning the surgeon's back and corresponds, approximately, to a prosthesis for the user's back which provides precise attaching and correction of postural habits shown to cause pain and muscle fatigue.

In the base of the torso support (1) there is a covered semi-hard surface, as well as the part of the attachment system to the torso support (1) that makes it possible to attach the posterior support (13). The semi-hard surface is divided in two parts and distinguishes the dorsal and lumbar regions, which are subsequently united by an elastomeric band attachment. This flexible joint allows controlled flexion and rotation movements while providing a stable support of the dorsal and lumbar regions, as illustrated in FIG. 11.

The posterior support (13) is comprised by a set of chained, perforated segments (13′). The segments (13′) are aligned by their format and they include an elastic medium (14) in their interior, the tension of which is controlled by an engine (15) placed in the seat support (4), located below the seat (3). The force exercised by the engine (15), and respective tension, is sustained in the posterior support (13) and controlled by the surgeon through the pressure from the surgeon's lower limbs on the tensioner pedal (19) located in the base (11). To increase the posterior support (13) tension, press the tensioner pedal (19) with the plus sign; to decrease the tension, press the tensioner pedal (19) with the minus sign. To attach or release the torso support (1) from the posterior support (13), the surgeon will press, using the lower limbs, the attach/release pedal (10) once. The device has a safety switch (9) and a remote control that can be used by a surgeon's assistant if the circumstances prevent the surgeon from making the adjustment. The safety switch (9) must be pressed when activation of any of the features available on the device is intended.

The posterior support (13) is attached to the seat support (4), the top of the actuators, the engine (15), the shaft (5), the lever (16) and the support actuator (6).

The device has a base structure (11) around which the weight of the various components that constitute it is distributed. Positioned in the centre of the base (11) is a hydraulic suspension (7) with the objective of relieving the pressure exercised on the actuators. Positioned on top of the hydraulic suspension (7) is the actuator support (6) which, in addition to supporting the linear actuator B (18), is one of the parts of the locking system between the base (11) and the seat support (4). The base (11) is also divided into two areas: the first which relies on the main linear actuator A (17) and the second where the battery (8) will be housed. This set, the hydraulic suspension (7), the linear actuator A (17) and the linear actuator B (18), are installed between the base (11) and the seat support (4) and are protected by a sleeve (20) in the connection between the seat (3) base and the fairing A (21) and fairing B (22). The device also comprises at the base (11), a caster set (12) that allows to move the device in the horizontal plane.

### Modes of Use

The device has two operating modes: the attached mode and the unattached mode.

In the attached mode, the surgeon is, imperatively, seated on the device and the torso support (1) is attached to the posterior support (13). In this mode, the user has a tensioner pedal (19) on the device's base that allows them to adjust the tension maintained by posterior support (13), relieving the surgeon of the effect of gravity. The attachment of the torso support (1) to the posterior support (13) is provided by a magnetic mechanism on the surface of the torso support (1) and the posterior support (13), activated by the passage of electricity in posterior support's biggest surface (13). Even when attached, the surgeon maintains near normal movements, being allowed moderate flexion, lateral extension and rotation. When the surgery is complete, the surgeon can release himself from the posterior support (13) by activating the safety switch and then pressing the attach/release pedal (10).

In the detached mode, the torso support (1) attached to the surgeon is not attached to the posterior support (13), allowing the user to move their upper body freely and to stand up from the device. Also in this mode, it is possible to use the posterior support (13) as a support surface, to sit back and rest, in the same way as using a common body support.

The present embodiment is naturally not in any way restricted to the embodiments described herein and a person of ordinary skill in the art can provide many modification possibilities thereof without departing from the general idea, as defined in the claims. The preferred embodiments described above are of course combinable with one another. The following claims further define preferred embodiments.

1. Body support device for surgery including a torso support (1), a vest (2), a seat (3), a posterior support (13), a seat support (4) and a base (11) characterized by the aforementioned torso support (1) being attached to the aforementioned posterior support (13) through means of a non-permanent connection and the torso support (1) position being controlled by the user with the use of an engine (15).

2. Body support device according to claim 1, wherein the posterior support (13) comprises by a set of longitudinally perforated segments (13′) with a concave spherical cap at the top and a convex spherical cap in the base.

3. Body support device according to any of the previous claims, wherein the posterior support (13) comprises by a set of segments (13′) chained and aligned by the passage of an elastic medium (14) on the inside and whose end is attached to a shaft (5), connected to an engine (15) through a belt (23).

4. Body support device according to any of the previous claims, wherein the user controls the attachment or release of the torso support (1) to the posterior support (13) through an attach/release pedal (10) located in the base that activates the non-permanent means of connection.

5. Body support device according to any of the previous claims, wherein the user controls the position of the torso support (1) through a tensioner pedal (19) located at the base, which controls the engine (5).

6. Body support device according to any of the previous claims, comprising a safety system activated through a safety switch pedal (9) located in the base that emits a beeping sound for a period of time during which the adjustments to the torso support (1) are allowed and, after the end of this beeping sound, the position of the device is locked, it being necessary to push the safety switch (9) to readjust its position.

7. Body support device according to any of the previous claims, comprising two linear actuators for the height and inclination of the seat (3).
